(19) United States
(12) Patent Application Publication
(10) Pub. No.: US 2014/0163361 A1
(43) Pub. Date: Jun. 12, 2014

(54) Combination Rotational and Phased-Array In Vivo Imaging Devices and Methods

(71) Applicant: Volcano Corporation, San Diego, CA (US)

(72) Inventors: Jeremy Stigall, Carlsbad, CA (US); Maritess Minas, San Diego, CA (US)

(21) Appl. No.: 14/103,297

(22) Filed: Dec. 11, 2013

Related U.S. Application Data
(60) Provisional application No. 61/736,468, filed on Dec. 12, 2012.

(51) Int. Cl. A61B 5/00; A61B 8/12

(52) U.S. Cl. CPC A61B 5/0035; A61B 5/0073; A61B 5/0059; A61B 8/12
USPC 600/427; 600/407; 600/459; 600/473; 600/463; 29/825

(57) ABSTRACT

An in vivo imaging device including at least two imaging modalities. In one aspect, the device includes a rotational imaging system in combination with a non-rotational imaging system. Systems including the imaging device, and methods of forming and in vivo imaging using a flexible, elongate body that includes two imaging modalities are also disclosed.

### CROSS-REFERENCE TO RELATED APPLICATION

The present application claims the benefit of the filing date of provisional U.S. Patent Application No. 61/736,468 filed Dec. 12, 2012. The entire disclosure of this provisional application is incorporated herein by this reference.

### TECHNICAL FIELD

The present disclosure relates generally to imaging inside the living body and, in particular, to an intravascular flexible, elongate imaging device that includes alternative imaging modalities including a rotational imaging system, e.g., ultrasound, and a phased-array imaging system. Methods of using the flexible, elongate imaging device, including toggling between modalities and concurrently operating both modalities, are also included.

### BACKGROUND

Intravascular ultrasound (IVUS) and other imaging techniques are widely used in interventional cardiology as a diagnostic tool for assessing a diseased vessel, such as an artery, within the human body to determine the need for treatment, to guide the intervention, and/or to assess its effectiveness. IVUS imaging uses ultrasound echoes to form a cross-sectional image of the vessel of interest. Typically, the ultrasound transducer on an IVUS catheter both emits ultrasound pulses and receives the reflected ultrasound echoes. The ultrasound waves pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound echoes to produce a cross-sectional image of the vessel where the catheter is located. Each type of IVUS can be used, for example, to characterize plaque in a patient's vessels. See, e.g., U.S. Publication No. 2003/0236443.

There are two types of IVUS catheters in common use today: solid-state and rotational, with each having advantages and disadvantages. Solid-state IVUS catheters use an array of ultrasound transducers (typically 64) distributed around the circumference of the catheter and connected to an electronic multiplexer circuit. The multiplexer circuit selects array elements for transmitting an ultrasound pulse and receiving the echo signal. By stepping through a sequence of transmit-receive pairs, the solid-state IVUS system can synthesize the effect of a mechanically scanned transducer element, but without moving parts. Since there is no rotating mechanical element, the transducer array can be placed in direct contact with the blood and vessel tissue with minimal risk of vessel trauma and the solid-state scanner can be wired directly to the imaging system with a simple electrical cable and a standard detachable electrical connector. Exemplary solid-state IVUS systems, also referred to as phased-array imaging systems, are marketed by Volcano Corporation and various such systems are described, for example, in U.S. Pat. No. 6,283,920 and U.S. Pat. No. 6,283,921. Such solid-state systems typically have lower resolution but higher depth of penetration than rotational systems, which can generate higher resolution images without losing wire positioning but tend to have lesser penetration depth into the vessel being imaged.

In the typical rotational IVUS catheter, a single ultrasound transducer element fabricated from a piezoelectric ceramic material is located at the tip of a flexible driveshaft that spins inside a plastic sheath inserted into the vessel of interest. The transducer element is oriented such that the ultrasound beam propagates generally perpendicular to the axis of the catheter. The fluid-filled sheath protects the vessel tissue from the spinning transducer and driveshaft while permitting ultrasound signals to freely propagate from the transducer into the tissue and back. As the driveshaft rotates (typically at 30 revolutions per second), the transducer is periodically excited with a high voltage pulse to emit a short burst of ultrasound. The same transducer then listens for the returning echoes reflected from various tissue structures, and the IVUS imaging system assembles a two dimensional display of the vessel cross-section from a sequence of several hundred of these ultrasound pulse/echo acquisition sequences occurring during a single revolution of the transducer. Rotational IVUS systems are marketed in the U.S., for example, by Volcano Corporation of San Diego, Calif., and are described, for example, in U.S. Pat. No. 6,221,015 and U.S. Patent Publication Numbers 2010/0234736 and 2010/0160788.

While the solid-state IVUS catheter is simple to use, thanks to its lack of moving parts, it cannot currently match the image quality available from a rotational IVUS catheter. It is difficult to operate a solid-state IVUS catheter at the same high frequency as a rotational IVUS device, and the lower operating frequency of solid-state IVUS catheters translates into poorer resolution compared to that of a higher frequency rotational IVUS catheter. There are also artifacts such as sidelobes, grating lobes, and poor elevation focus (perpendicular to the imaging plane) that arise from the array-based imaging that are greatly reduced or completely absent with a rotational IVUS device. Despite the image quality advantages of the rotational IVUS catheter, each of these devices has found a niche in the interventional cardiology market, with solid-state IVUS preferred in circumstances where ease-of-use is paramount and the reduced image quality is acceptable for the particular diagnostic needs, while rotational IVUS is preferred where image quality is paramount and the more time-consuming catheter preparation is justified.

In the rotational IVUS catheter, the ultrasound transducer is typically a piezoelectric ceramic element with low electrical impedance capable of directly driving an electrical cable connecting the transducer to the imaging system hardware. In this case, a single pair of electrical leads (or coaxial cable) is used to carry the transmit pulse from the system to the transducer and to carry the received echo signals from the transducer back to the imaging system by way of a patient interface module, where they are assembled into an image. An important complication in this electrical interface is the transportation of electrical signals across a rotating mechanical junction. Since the catheter driveshaft and transducer are spinning (in order to scan a cross-section of the artery) and the imaging system hardware is stationary, there must be an electromechanical interface where the electrical signals traverse the rotating junction. In rotational IVUS imaging systems, this problem can be solved by a variety of different approaches, including the use of a rotary transformer, slip rings, rotary capacitors, etc.

While existing catheters deliver useful diagnostic information, there is a need for enhanced image quality and ease of use to provide more valuable insight into the condition of vessels and passageways in vivo. Accordingly, there remains a need for improved devices, systems, and methods for providing a superior imaging device compared to those presently available.

### SUMMARY

Embodiments of the present disclosure provide a combination rotational in vivo imaging system and a phased-array imaging system compactly packaged in a single flexible, elongate imaging package, such as a catheter, for delivery to a diagnostic zone in a patient (e.g., a person being diagnosed) to advantageously provide a benefit from having both types of imaging system available while minimizing one or more disadvantages of a given imaging modality. A health care practitioner or other user is thus provided multiple choices in visualizing abnormalities in the coronary arteries or other patient vessels or passageways requiring imaging, as well as the simplicity of phased array imaging with the accuracy and clarity of a rotational imaging device.

In a first aspect, the present disclosure encompasses an in vivo imaging device including at least two imaging modalities, which are preferably different. The device includes a flexible, elongate body having a proximal portion and a distal portion, where the flexible, elongate body further includes: a first imaging element secured proximal to the distal tip; a second imaging element that provides rotational imaging and is secured proximal to the distal tip and the first imaging element; a lumen that extends at least partially along the length of the flexible, elongate body and that encompasses at least a portion of the second imaging element; and a second lumen extending along the length of the flexible, elongate body that encompasses a plurality of electrically conductive connectors associated with the first imaging element.

In one embodiment, the first imaging element includes an ultrasound transducer, which can be an intravascular ultrasound (IVUS) transducer. In a preferred embodiment, the transducer includes an array of solid-state ultrasound transducer elements. In another embodiment, the first imaging element includes at least a portion of an optical coherence tomography (OCT) device including an optical fiber or reflector. In yet another embodiment, the second imaging element includes at least one ultrasound transducer, at least one infrared transmission element, or at least a portion of an optical coherence tomography (OCT) device including an optical fiber or reflector.

In another embodiment, the lumen is in communication with an opening in a sidewall of the flexible, elongate body to allow fluid flow through the lumen. In a further embodiment, the device further includes a distal lumen that extends from the distal tip and proximal to the first imaging element and that is configured to receive a guide wire. In a preferred embodiment, the guide wire is configured to exit at an end of the distal lumen that is distal from the second imaging element.

In a preferred embodiment, the device further includes an application-specific integrated circuit (ASIC) coupled to the distal portion of the flexible, elongate member, wherein the ASIC is electrically coupled to at least one of the imaging elements and wherein the ASIC includes: a pulser for driving transmitted signals from the at least one imaging element; an amplifier for receiving and amplifying signals representative of reflected signals received by the at least one imaging element; a protection circuit configured to protect the amplifier from high voltage transmit pulses from the pulser and allow the amplifier to receive the low amplitude echo signals from the at least one imaging element; and timing and control circuitry for coordinating operation of the pulser, amplifier, and protection circuit.

In a second aspect, the present disclosure encompasses a method of in vivo imaging of a patient's tissue which includes: introducing a flexible, elongate imaging device having a proximal end and a distal tip end including at least two imaging modalities including a first imaging element secured proximal to the distal tip end, and a second imaging element that provides rotational imaging and is secured proximal to the distal tip end and the first imaging element; advancing the imaging device to a position immediately adjacent a tissue zone to be imaged such that a distal tip of the imaging device is at least adjacent to the tissue zone to be imaged; and obtaining one or more images of the tissue zone using at least one of the first or second imaging elements.

In a further embodiment, after the first imaging element obtains one or more images of the zone, the flexible, elongate imaging device is then advanced to position the second imaging element more closely adjacent the zone, before obtaining one or more additional images of the zone. In a preferred embodiment, the flexible, elongate imaging device is selected to include a catheter. In yet another preferred embodiment, the imaging device is advanced to a position adjacent the zone of the tissue such that the at least first imaging element is within about 5 mm of the zone of the tissue. In a more preferred embodiment, the imaging device is advanced to a position adjacent the zone of the tissue such that the at least first imaging element is within about 3 mm of the zone.

In a third aspect, the disclosure encompasses an in vivo imaging system including the imaging device discussed above; an interface module configured to connect with the proximal connector of the imaging device; and an image processing component in communication with the interface module. The in vivo imaging system may be configured for intravascular, respiratory (including nasal, esophageal, etc.), and other tissues, or a combination thereof.

In a fourth aspect, the disclosure encompasses a method of forming an in vivo imaging device which includes providing a flexible, elongate body having a proximal portion and a distal portion having a distal tip and disposing within the flexible, elongate body an imaging system which includes: a first imaging element secured proximal to the distal tip; a second imaging element that provides rotational imaging and is secured proximal to the distal tip and the first imaging element; a lumen extending at least partially along the length of the flexible, elongate body that encompasses at least a portion of the second imaging element; and a second lumen extending along the length of the flexible, elongate body that encompasses a plurality of electrically conductive connectors associated with the first imaging element. In a preferred embodiment, the in vivo imaging device is configured for intravascular imaging.

In one embodiment, the method further includes providing the flexible, elongate body so as to have at least a substantially constant diameter along a majority of its length between the proximal and distal portions; and providing a tapered portion to the distal tip so as to taper from the at least substantially constant diameter of the flexible, elongate body to a smaller diameter as the distal tip extends distally along a longitudinal axis of the flexible, elongate body. In a preferred embodiment, the tapered portion of the distal tip has a length less than about 5 mm.

Additional aspects, features, and advantages of the present disclosure will become apparent from the following detailed description.

### BRIEF DESCRIPTION OF THE DRAWINGS

The present disclosure is best understood from the following detailed description when read with the embodiments, or examples, illustrated in the accompanying figures. It is emphasized that various features are not necessarily drawn to scale. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

Illustrative embodiments of the present disclosure, which form part of the present specification, will be described with reference to the accompanying drawings, of which:

FIG. 1 is a diagrammatic schematic view of an imaging system according to an embodiment of the present disclosure.

FIG. 2 is a diagrammatic, partial cutaway side view of a distal portion of an imaging device according to an embodiment of the present disclosure.

FIG. 3 is a cross-sectional view of the side view of a proximal portion of the imaging device shown in FIG. 2, according to an embodiment of the present disclosure.

FIG. 4 is a cross-sectional view of the side view of a distal portion of the imaging device shown in FIG. 2, according to an embodiment of the present disclosure.

FIG. 5 is a diagrammatic, partial cutaway side view of the distal tip portion of the imaging system shown in FIG. 1.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Specific examples of components and arrangements are described below to simplify the present disclosure. These are, of course, merely examples and are not intended to be limiting. Any alterations and further modifications to the described devices, systems, and methods, and any further application of the principles of the present disclosure are fully contemplated and included within the present disclosure as would normally occur to one of ordinary skill in the art to which the disclosure relates. In particular, it is fully contemplated that the features, components, and/or methods described with respect to one embodiment may be combined with the features, components, and/or methods described with respect to other embodiments of the present disclosure. For the sake of brevity, however, the numerous iterations of these combinations will not be described separately.

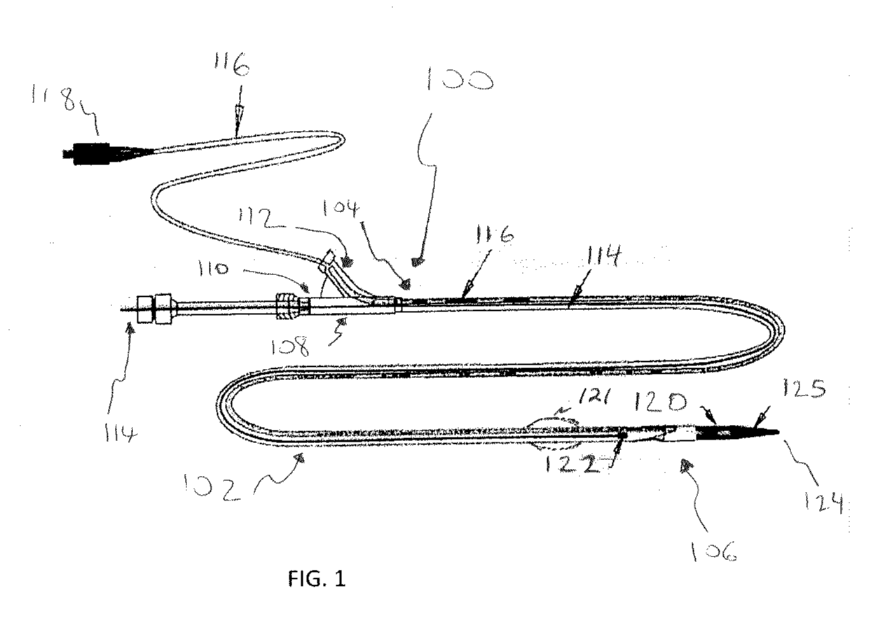

Referring to FIG. 1, shown therein is an imaging device 100 according to an embodiment of the present disclosure that includes two imaging elements towards the distal portion 106. As shown, the imaging device 100 comprises an elongate, flexible body 102 having a proximal portion 104 and a distal portion 106. The proximal portion 104 includes an adapter 108. In the illustrated embodiment, the adapter 108 is y-shaped with extensions 110 and 112. In that regard, extension 110 generally extends along the longitudinal axis of the body 102, while extension 112 extends at an oblique angle with respect to the longitudinal axis of the body. Generally, the extensions 110 and 112 provide access to the flexible, elongate body 102. In the illustrated embodiment, extension 110 is configured to receive a lumen 114 that is sized and shaped to encompass a second imaging element 120, and that extends along the length of the body 102 from the proximal portion 104 to the distal portion 106 and defines an opening towards the distal end of the imaging device 100. In some embodiments, the lumen 114 of the imaging device 100 is centered about the central longitudinal axis of the body 102. In preferred embodiments, however, the lumen is offset with respect to the central longitudinal axis of the body 102. In an exemplary embodiment, the lumen 114 is offset, e.g., to provide sufficient space in the body 102 for a second lumen 116.

In the illustrated embodiment, extension 112 of adapter 108 is configured to receive communication lines (e.g., electrical, optical, and/or combinations thereof) that are coupled to a first imaging component 120 positioned within the distal portion 106 of the imaging device 100. In that regard, a second lumen 116 containing one or more communication lines extends from extension 112 to a connector 118. The connector 118 is configured to interface the imaging device directly or indirectly with one or more of a patient interface module (“PIM”), a processor, a controller, and/or combinations thereof (not shown). The particular type of connection depends on the type of imaging components 120, 122 implemented in the imaging device, but generally include one or more of an electrical connection, an optical connection, and/or combinations thereof. The second imaging component 122 may also include a connection through these connectors, in addition to or instead of lumen 114. In a preferred embodiment, the first imaging component 120 involves a plurality of electrical connections in the second lumen 116. The second lumen 116 for the plurality of electrically conductive connectors can in certain embodiments be partially embedded or entirely overmolded, i.e., defined by molding the second lumen 116 itself or just the electrical connectors themselves into the flexible, elongate body 102. In the latter embodiment, the electrical connectors define their own second lumen 116 embedded in the wall of the body 102. In all embodiments, first and second lumens 114 and 116 extend from a proximal end of the body 102 to the distal portion 106 as further discussed herein.

In an optional embodiment (not shown), the distal portion 106 may include one or more markers (not shown) that are visible using non-invasive imaging techniques (e.g., fluoroscopy, x-ray, CT scan, etc.) to track the location of the distal portion 106 of the imaging device 100 within a patient. Accordingly, in some instances the markers may be radiopaque bands extending around the circumference of the body 102. Further, the marker(s) can be positioned at known, fixed distances from the first imaging component 120 and/or the distal end 124 of the imaging device 100 in some instances. Further, in some embodiments, one or more components associated with the first imaging component 120 can be used as a marker to provide a reference of the position of the distal portion 106 of the imaging device 100. Still further, the system may include an external balloon 121 associated with or carried on the exterior of the flexible, elongate body 102. For certain imaging modalities, such as OCT and high-resolution ultrasound, the balloon may be inflated to provide an evacuated area that reduces and preferably eliminates blood temporarily to enhance image clarity.

The first imaging component 120 is adjacent the distal end 124 of the flexible, elongate body 102 and may be any type of imaging element suitable for in vivo visualization, e.g., intravascular. Accordingly, the first imaging component 120 may include an ultrasound transducer array (e.g., arrays having 16, 32, 64, or 128 elements are utilized in varying embodiments), a single ultrasound transducer, or one or more optical coherence tomography (“OCT”) or infrared (IR) elements (e.g., mirror, reflector, and/or optical fiber), and/or combinations thereof. Preferably, the first imaging component 120 is solid state and is secured to the body 102 at a distal location. As depicted, the body 102 includes a distal tip 125 that tapers towards the distal end 124. In another embodiment (not shown), no tip is present and the body 102 simply truncates in any desired shape, such as a rounded tip or flat tip. The first imaging component 120 can preferably be tilted within a range of angles to provide a view forward (in the distal direction) or backwards (in the proximal direction).

The second imaging component 122, which provides rotational imaging to provide a circumferential image of a patient's vessel, is disposed in the distal portion 106 of the flexible, elongate body 102 at a position proximal to the distal first imaging component 120. In that regard, in some embodiments the imaging device 100, or a drive cable disposed in first lumen 114 and mechanically associated with the second imaging component 122, is configured to be rotated (either manually by hand or by use of a motor or other rotary device) to obtain images of the inside of the vessel wall. The second imaging component 122 may include an ultrasound transducer, an ultrasound transducer array (e.g., arrays having 16, 32, 64, or 128 elements are utilized in varying embodiments), or one or more optical coherence tomography (“OCT”) or infrared (IR) elements (e.g., mirror, reflector, and/or optical fiber), and/or combinations thereof. Each of the first and second imaging components 120, 122 includes at least one transmitter or pulser to produce the relevant signal, as well as at least one detector to receive the reflected signal. In the preferred embodiment depicted, first imaging component 120 includes an ultrasound transducer array of 64 elements and second imaging component 122 includes a rotational ultrasound transducer. When an array of transmitters or pulsers, e.g., of ultrasound, is included, the image data from each element is combined to form a circumferential image of the patient's vessel.

In one embodiment, the flexible, elongate body 102 has at least a substantially constant diameter along a majority or all of its length between the proximal and distal portions 104, 106. By substantially constant is meant fewer than about a 20% variation in diameter, preferably less than about a 10% variation in diameter, from the narrowest to widest diameter along the length of the flexible, elongate body 102. Typically, the less variation (i.e., smoother) the diameter, the less risk of the flexible, elongate body 102 chafing, irritating, or even being caught on a protrusion in a patient's vessel when inserted or removed to position the distal portion 106 for imaging a desired zone in the vessel. In certain embodiments, the distal portion 106 defines a distal tip 125 that tapers from the at least substantially constant diameter of the flexible, elongate body 102 to a smaller diameter as the distal tip 125 extends distally along a longitudinal axis of the flexible, elongate body 102. Other shapes may be employed in alternative embodiments. The tapered portion of the distal tip 125, when included, may have a length less than about 10 mm, preferably less than about 5 mm.

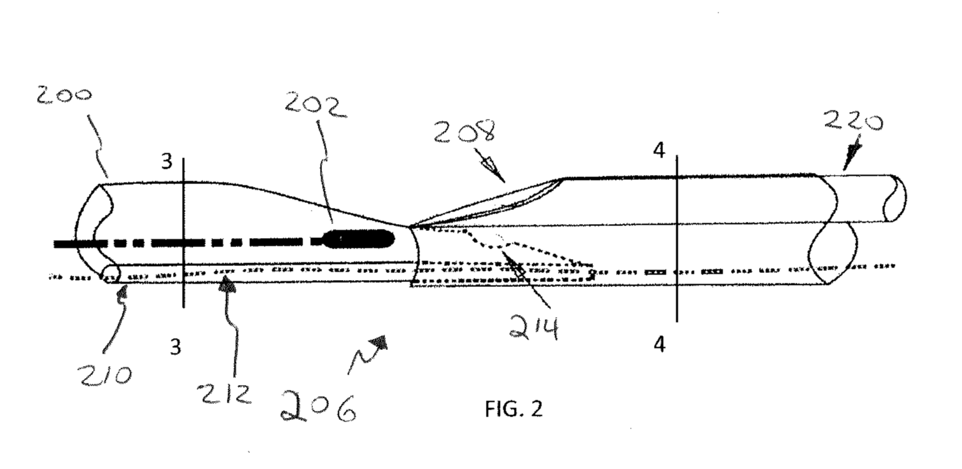

Referring now to FIG. 2, a diagrammatic, partial cutaway side view of a distal portion 206 of an imaging device according to an embodiment of the present disclosure is shown. As shown, first lumen 200 extends at least partially along the length of the encompassing flexible, elongate body (not shown) and encompasses at least a portion of a second imaging element 202 that provides rotational imaging. The second imaging element 202 may include a drive cable, visual or electrical connectors to transmit signals back in the proximal direction, etc. and terminates adjacent an optional but preferred exit port 208, such as for a guide wire. At its distal end, second imaging element 202 includes the rotational imaging elements described herein. A second lumen 210 extends along the length of the encompassing flexible, elongate body (not shown) to carry one or more connectors 212 associated with the first imaging element (not shown) that is disposed in a more distal location. The connector(s) 112 are preferably electrically conductive to carry signals between the first imaging element (not shown) in the distal direction and an interface in a proximal direction. As shown, the second lumen 210 terminates at a position distal to the distal end of the first lumen 200, although the second lumen 210 could be extend even further or even be mechanically associated with the first imaging device (not shown).

FIG. 2 also includes an optional but preferred third lumen 220 that extends from the exit port 208 in a distal direction, which can be used in association with the guide wire. As also shown, a flush port 214 may be provided in adjacent an end of the first lumen 200, which can facilitate sterilization such as through a saline flush, for example, when the device is not in use. The flush port 214 may also be used to inject saline to flush out the air and fill the distal portion 106 of the flexible, elongate body 102 with an ultrasound-compatible fluid at the time of use of the imaging device. The saline or other similar flush is typically required since air does not readily conduct ultrasound. Saline also provides a biocompatible lubricant for the rotating driveshaft.

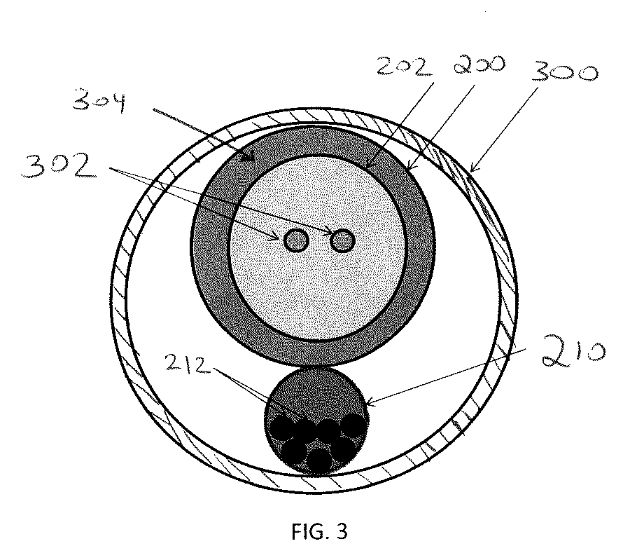

FIG. 3 depicts a cross-sectional view of the side view of a proximal portion of the imaging device shown in FIG. 2, according to an embodiment of the present disclosure. In certain situations, the features or functionality are similar or identical to those discussed before, and in such cases the same reference numerals have been used to refer to analogous features. Here, the flexible, elongate body 300 is shown encompassing the first and second lumens 200, 210. The first lumen 200 carries the connectors for the second imaging device 202, including a drive and additional connectors 302 that extend between the transmitter and detector components at the distal end and operatively connect to the interface in a proximal direction. The first lumen 200 as shown also includes a fluid 304. The fluid 304 may serve various purposes, including minimizing friction from the rotational drive component 202 against the first lumen 200, providing a cooling effect from the heat caused by the second imaging device 202, etc. As depicted, second lumen 210 is entirely inside the flexible, elongate body 300 rather than partially embedded or overmolded in the body 300. The second lumen 210 encompasses the one or more connectors 212. As depicted, seven (7) electrical connectors 212 may be used to connect to a first imaging component at a distal end of the flexible, elongate body 300 and an interface in a proximal direction.

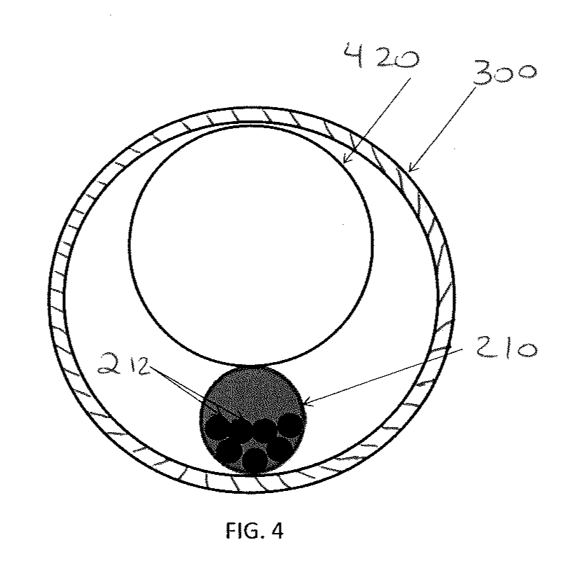

FIG. 4 is a cross-sectional view of the side view of a distal portion of the imaging device shown in FIG. 2, according to an embodiment of the present disclosure. At this cross-section, the second lumen 210 is still present including the connector(s) 212 encompassed by the flexible, elongate body 300. The third lumen 420 is present here, and is available for example to contain a portion of a guide wire that may be used to position the flexible, elongate body 300 in a patient's vessel at a zone or zones to be imaged. The third lumen 420 can form a portion of a rapid exchange guide wire tracking configuration.

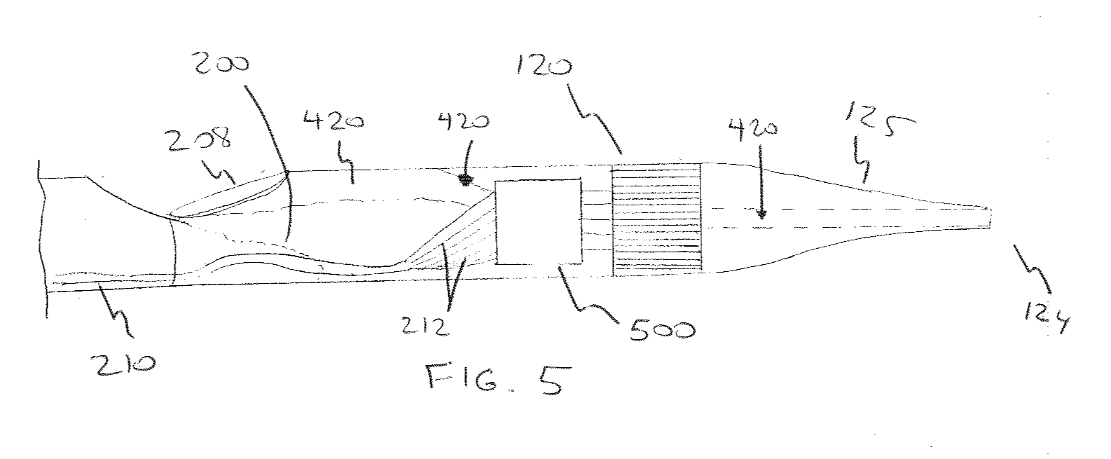

FIG. 5 is a diagrammatic, partial cutaway side view of the distal tip portion of the imaging system shown in FIG. 1. As shown, the distal end 124 has a tapered tip 125 that tapers in a direction from a proximal side to the distal end 124. The second lumen 210 extends from a proximal direction past a distal end of the first lumen 200 and towards the first imaging component 120. As shown, the second lumen 210 ends sufficiently before the first imaging component 120 so that the plurality of connectors 212 therein can extend distally to their connection points on an application-specific integrated circuit (ASIC) 500. The ASIC 500 is electrically connected to the first imaging component 120, which as shown is a phased-array ultrasound transducer to emit and detect ultrasound signals reflected back from the vessel zone being imaged. The first imaging component 120 may be any of the suitable transmitter/detector pairings discussed herein. The third lumen 420 noted on FIG. 4 is shown here to extend from an exit port 208 in a proximal location distally toward a longitudinal axis of the flexible, elongate body 102 to minimize any imaging artifacts, and distally past the first imaging component 120 to the distal end 124. This can advantageously permit a guide wire to be inserted at the exit port 208 and extend into the distal tip 125 to facilitate positioning of the flexible, elongate body 102.

In one embodiment, the first imaging component 120 is spaced from the distal end 124 of the flexible, elongate body 102 by a distance of about 10 mm or less, preferably about 5 mm or less. The first imaging component 120 is typically secured in fixed position to the flexible, elongate body 102 as well, to increase the accuracy of imaging.

Additional Embodiments of the Apparatus and Its Operation

The flexible, elongate body having a proximal portion and a distal portion is preferably a catheter. It should be understood that any available transmitter and detector device(s) may be used as the first and second imaging components. In a preferred embodiment, the first imaging device is a fixed, phased-array transmitter and detector and the second imaging device is a rotational imaging device. Preferably, these are each independently selected as an ultrasound-based imaging device.

One of the lumens is preferably in communication with an opening in a sidewall of the flexible, elongate body such that the imaging device is configured as a rapid-exchange catheter, although various configurations can be envisioned. The flexible, elongate body preferably includes a distal lumen that extends from the distal tip and proximal to the first imaging element. This distal lumen is configured to receive a guide wire. Because the guide wire can create imaging artifacts, it is best placed centrally along a longitudinal axis of the flexible, elongate body to minimize or avoid interference particularly with the first imaging element adjacent the distal tip. In this embodiment, the guide wire is configured to exit at an end of the distal lumen that is distal from the second imaging element.

Various arrangements of connectors, particularly electrical and/or visual, can be envisioned based on the disclosure herein. For example, the imaging device may include one or more application-specific integrated circuits (ASICs) at the distal portion of the flexible, elongate member, wherein an ASIC is electrically coupled to at least one of the imaging elements and wherein each ASIC includes timing and control circuitry for coordinating operation of the transmitter(s), such as in a phased-array, and the one or more detectors to receive reflected signals. Each ASIC may preferably include the following:

an transmitter (e.g., a pulser) for driving transmitted signals from the at least one imaging element,

optionally, but preferably, an amplifier for receiving and amplifying signals representative of reflected signals received by the at least one imaging element,

optionally, but preferably, a protection circuit configured to protect the amplifier from high voltage transmit pulses from the pulser and allow the amplifier to receive the low amplitude echo signals from the at least one imaging element, and

timing and control circuitry for coordinating operation of the pulser, amplifier, and optional protection circuit. Alternatively, an ASIC may provide the timing and control circuitry, an optional amplifier and/or optional protection circuit, and be electrically associated with the first and second imaging elements that provide the emitting and detecting of received signals.

It should be understood that multiple ASICs may be used, such as one for each of the first and second imaging devices, or two in parallel to provide redundancy if space in the flexible, elongate body permits. Embodiments of the present disclosure implement more elaborate protection schemes that use active elements (e.g., transistors) to implement the protection functions. Such active protection circuits can be more efficient and more readily implemented on an ASIC. One embodiment of an active protection circuit can implement a high voltage analog switch circuit that is controlled by a timing circuit to open during the transmit pulse and to close during receiving of the ultrasound echo signals. One of the complications associated with this approach is that the timing signal that opens the switch during transmit pulse must be 100% reliable, since a single errant high voltage pulse could destroy the amplifier. This level of reliability is difficult to ensure when the timing, transmitter, and protection circuits are physically separated from one another. Accordingly, in some embodiments of the present disclosure the timing, transmitter, and protection circuits are closely coupled together within a single ASIC.

Another important aspect of certain embodiments of the present disclosure when an ASIC is present is to manage the power dissipation in the circuit to prevent excessive temperature rise at the distal end of the catheter where the ASIC is located. The largest source of power dissipation in the ASIC is amplifier circuit, which when included requires a relatively high bias current to provide the desired performance. One method to reduce the power consumption is to shut down the amplifier when it is not needed. Typically, there is a period of approximately 10 μsec after each transmit pulse for receiving ultrasound echoes, and a typical pulse repetition period for transmit pulses is about 60 μsec, resulting in an amplifier duty cycle as low as 16%. By placing the amplifier in a low power standby mode when it is not needed, the power (and consequent heat output) can be reduced to approximately one-sixth of what would be required for continuous operation. One option for controlling the amplifier shutdown is to include a timing circuit on the ASIC to enable the amplifier for a 10 μsec duration after each transmit pulse. While this approach is simple to implement and suitable for some applications, it lacks the flexibility to adapt to different transducer configurations or imaging modes that might demand a different receive duration. An alternative approach is to define a command protocol whereby one pulse sequence sent from the PIM to the ASIC triggers a transmit pulse, while a later pulse sequence triggers the termination of the receive window. In this fashion, the PIM can control the ASIC timing and the PIM can be easily programmed and/or reprogrammed to adjust the timing for each mode or transducer configuration. One example of a simple protocol is defined as follows: the first pulse sequence to be sent from the PIM after a long quiet spell (20 μsec, for example) would be interpreted as a transmit pulse sequence, and any subsequent pulse occurring within a 20 μsec window would be interpreted as terminating the receive window and rearming the transmitter to fire on the next pulse sequence. As one of ordinary skill in the art will appreciate, any number of various timing protocols may be utilized, depending on the particular transducer configuration and/or imaging mode.

The ability to manage the circuit power dissipation by controlling the amplifier duty cycle with a simple sequence of pulses as described previously adds flexibility to the system to address multiple applications. For greater flexibility, it may be desirable to add a higher degree of programmability to the ASIC, to enable a wider range of programmability in the circuit operation. This can be accomplished without greatly increasing the complexity of the device by defining a simple serial communication protocol to permit the PIM to send configuration information to the ASIC over the same two-wire communication link as used for the transmit trigger pulses and for optional receive window termination pulses. Examples of the type of configuration information that might be programmed into the circuit over the serial communications link include amplifier gain, amplifier bias current, transmit damping pulse duration, and/or other parameters.

The operation of the combination imaging device including at least the two imaging modalities of the present disclosure should be readily apparent to those of ordinary skill in the art with reference to the device and system discussed herein. For example, the imaging device described herein may be used to conduct intravascular imaging of one or more zones in a patient's vessel by introducing a flexible, elongate imaging device having a proximal end and a distal tip end including at least two imaging modalities comprising: a first imaging element secured proximal to the distal tip end; a second imaging element that provides intravascular rotational imaging and is secured proximal to the distal tip end and the first imaging element; advancing the imaging device to a position immediately adjacent a zone of the vessel to be imaged such that a distal tip of the imaging device is at least adjacent to the zone such that at least the first imaging element is spaced sufficiently closely to the vessel zone to be imaged that a reasonably accurate and clear image can be obtained; and obtaining one or more images of the zone using at least one of the first or second imaging elements. When the first imaging element is a phased-array system, such as intravascular ultrasound having a plurality of transducers, sufficiently close may refer to being within about 10 mm, and preferably within about 5 mm of the zone. In preferred embodiments, this distance may be within about 3 mm, or even within about 1 mm of the zone to be imaged.

The first and second imaging elements are independently selected to comprise an ultrasound device and ultrasound transducer, an optical coherence tomography device and an optical fiber or reflector, or an infrared device and an optical fiber or reflector. While any combination imaging device disclosed herein may be used to achieve such intravascular imaging, it may be preferred to select first and second imaging elements to use the same type of signal for ease of processing and analysis, e.g., ultrasound, optical coherence tomography, or infrared. In a preferred imaging method, the first and second imaging elements are each selected to include at least one intravascular ultrasound (IVUS) transducer with the second imaging element being a rotational arrangement as previously noted. Preferably, the first imaging element includes an array of solid-state ultrasound transducer elements. These can be any suitable array arrangement, such as including 16, 32, 64, or 128 elements in varying embodiments.

The operation of the combination imaging device to obtain one or more images will preferably include obtaining at least one image with either the first or second imaging element, and obtaining at least one image with the other imaging element. While only one imaging element need be used, the full advantages of the combination imaging device will involve obtaining images with each of the first and second imaging components. Preferably, this is achieved while the flexible, elongate body remains in situ, without having to remove the flexible, elongate body from a patient and then reinsert it to image the vessel zone of interest. In certain embodiments, one or more images will be obtained operating the first and second imaging components or elements sequentially in either order. In other embodiments, the first and second imaging components or imaging elements may be operated concurrently. One potential benefit of sequential operation is to minimize or avoid any potential interference in image quality caused when signals from one of the imaging components are received by the other imaging component. When concurrent operation is desired, it may thus be preferred to select first and second components to use different types of transmitters (e.g., a phased-array OCT as the first imaging device and a rotational ultrasound as the second imaging device) to minimize any potential interference or reduction in quality.

While the flexible, elongate body remains in situ, it may be used to take a series of images over time or advanced or withdrawn within a vessel to capture images of adjacent zones to obtain a more complete understanding of vessel of interest being evaluated. For example, in some embodiments, after the first imaging element obtains one or more images of the zone, the flexible, elongate imaging device is then advanced to position the second imaging element more closely adjacent the zone, before obtaining one or more additional images of the zone. It should be understood that by “advanced” is meant simply “moved,” which could be in a distal direction further into the vessel or in a proximal direction out of the vessel. Preferably, the flexible, elongate imaging device is a catheter, such as a rapid-exchange catheter.

It should be understood that the imaging device discussed herein may be associated with additional components and provided as an intravascular imaging system. For example, such a system could include the combination imaging device with a first imaging component and a rotational second imaging component discussed herein, operatively associated with an interface module (e.g., a PIM) configured to connect with the proximal connector of the imaging device; and an intravascular image processing component in communication with the interface module. An intravascular image processing component might include a computer or other electronic processor to process the electrical signals provided from the imaging devices or other electrically associated equipment, such as one or more ASICs. Preferably, the output of such processed signals is displayed for a user, who may be operating the equipment or merely observing/analyzing its operation and who may be proximate the equipment or remotely located. The output of processed signals is preferably also stored for later analysis or other uses.

The combination imaging device disclosed herein may be formed by any available method using any available techniques, components, or equipment, particularly with reference to the guidance herein provided. In a general embodiment, the intravascular imaging device may be formed by providing a flexible, elongate body having proximal portion and a distal portion having a distal tip, and disposing within the flexible, elongate body an imaging system which includes at least the following: a first imaging element secured proximal to the distal tip; a second imaging element that provides intravascular rotational imaging and is secured proximal to the distal tip and the first imaging element; a lumen extending at least partially along the length of the flexible, elongate body that encompasses at least a portion of the second imaging element; and a second lumen extending along the length of the flexible, elongate body that encompasses a plurality of electrically conductive connectors associated with the first imaging element.

It is typically desirable to provide the flexible, elongate body so as to have at least a substantially constant diameter along a majority of its length between the proximal and distal portions. In certain embodiments, a tapered portion can be provided to an optional distal tip so as to taper from the flexible, elongate body to a smaller diameter as the distal tip extends distally along a longitudinal axis of the flexible, elongate body.

In certain embodiments, it is preferred to include a compatible patient interface module (PIM), an imaging console or processing system, and a monitor to display the images generated by the imaging console. First and second imaging devices, as discussed above, may optionally but preferably include associated circuitry mounted near a distal tip of the flexible, elongated body, and the appropriate electrical connector(s) to support the PIM. The PIM can be arranged to generate the required sequence of transmit trigger signals and control waveforms to regulate the operation of the circuit(s), and to process any amplified echo signals received. The PIM also may provide, in certain embodiments, high- and low-voltage DC power supplies to support operation of the first or second imaging components, or both. An important feature of the PIM is that it must deliver DC supply voltages to the circuitry of the flexible, elongated body across a rotational interface for the second imaging device. In such embodiments, slip-rings and/or the implementation of the active spinner technology described in U.S. Patent Application Publication No. 2010/0234736, which is hereby incorporated by reference in its entirety, may be used in place of a rotary transformer that is more typical in embodiments with AC power.

Persons of ordinary skill in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An in vivo imaging device including at least two different imaging modalities, which device comprises:
a flexible, elongate body having a proximal portion and a distal portion, where the flexible, elongate body further comprises:
a first imaging element secured proximal to the distal tip;
a second imaging element that provides intravascular rotational imaging and is secured proximal to the distal tip and the first imaging element;
a lumen that extends at least partially along the length of the flexible, elongate body and that encompasses at least a portion of the second imaging element; and
a second lumen extending along the length of the flexible, elongate body that encompasses a plurality of electrically conductive connectors associated with the first imaging element.

2. The imaging device of claim 1, wherein the first imaging element comprises at least one ultrasound transducer.

3. The imaging device of claim 2, wherein the transducer comprises an array of solid-state ultrasound transducer elements.

4. The imaging device of claim 1, wherein the first imaging element comprises at least a portion of an optical coherence tomography device including an optical fiber or reflector.

5. The imaging device of claim 1, wherein the second imaging element comprises at least one of an ultrasound transducer, an infrared transmission element, or an optical coherence tomography device including an optical fiber or reflector.

6. The imaging device of claim 1, wherein the lumen is in communication with an opening in a sidewall of the flexible, elongate body to allow fluid flow through the lumen.

7. The imaging device of claim 1, which further comprises a distal lumen that extends from the distal tip and proximal to the first imaging element and that is configured to receive a guide wire.

8. The imaging device of claim 7, wherein the guide wire is configured to exit at an end of the distal lumen that is distal from the second imaging element.

9. The device of claim 1, which further comprises an application-specific integrated circuit (ASIC) coupled to the distal portion of the flexible, elongate member, wherein the ASIC is electrically coupled to at least one of the imaging elements and wherein the ASIC includes:
a pulser for driving transmitted signals from the at least one imaging element,
an amplifier for receiving and amplifying signals representative of reflected signals received by the at least one imaging element,
a protection circuit configured to protect the amplifier from high voltage transmit pulses from the pulser and allow the amplifier to receive the low amplitude echo signals from the at least one imaging element, and
timing and control circuitry for coordinating operation of the pulser, amplifier, and protection circuit.

10. A method of in vivo imaging of a patient which comprises:
introducing a flexible, elongate imaging device having a proximal end and a distal tip end including at least two imaging modalities comprising:
a first imaging element secured proximal to the distal tip end;
a second imaging element that provides rotational imaging and is secured proximal to the distal tip end and the first imaging element;
advancing the imaging device to a position immediately adjacent a tissue zone to be imaged such that a distal tip of the imaging device is at least adjacent to the zone to be imaged; and
obtaining one or more images of the tissue zone using at least one of the first or second imaging elements.

11. The method of claim 10, wherein the first and second imaging elements are independently selected to comprise an ultrasound transducer, an optical coherence tomography device including an optical fiber or reflector, or an infrared transmission element having an optical fiber or reflector.

12. The method of claim 10, wherein the first and second imaging elements are each selected to comprise an intravascular ultrasound (IVUS) transducer.

13. The method of claim 12, wherein the first imaging element comprises an array of solid-state ultrasound transducer elements.

14. The method of claim 10, wherein the obtaining one or more images comprises obtaining at least one image with either the first or second imaging element, and obtaining at least one image with the other imaging element.

15. The method of claim 14, wherein the obtaining one or more images with the first and second imaging elements occurs sequentially in either order.

16. The method of claim 14, wherein the obtaining one or more images using the first and second imaging elements is achieved while the flexible, elongate imaging device remains in vivo.

17. The method of claim 10, wherein, after the first imaging element obtains one or more images of the tissue zone, the flexible, elongate imaging device is then advanced to position the second imaging element more closely adjacent the tissue zone, before obtaining one or more additional images of the zone.

18. The method of claim 10, wherein the flexible, elongate imaging device is selected to comprise a catheter.

19. The method of claim 10, wherein the imaging device is advanced to a position adjacent the tissue zone such that the at least first imaging element is within about 5 mm of the tissue zone.

20. The method of claim 10, wherein the imaging device is advanced to a position adjacent the tissue zone such that the at least first imaging element is within about 3 mm of the tissue zone.

21. An in vivo imaging system comprising:
the imaging device of claim 1;
an interface module configured to connect with the proximal connector of the imaging device; and
an image processing component in communication with the interface module.

22. A method of forming an intravascular imaging device which comprises:
providing a flexible, elongate body having proximal portion and a distal portion having a distal tip; and
disposing within the flexible, elongate body an imaging system which comprises:
a first imaging element secured proximal to the distal tip;
a second imaging element that provides intravascular rotational imaging and is secured proximal to the distal tip and the first imaging element;
a lumen extending at least partially along the length of the flexible, elongate body that encompasses at least a portion of the second imaging element; and
a second lumen extending along the length of the flexible, elongate body that encompasses a plurality of electrically conductive connectors associated with the first imaging element.

23. The method of claim 22, which further comprises: providing the flexible, elongate body so as to have at least a substantially constant diameter along a majority of its length between the proximal and distal portions; and providing a tapered portion to the distal tip so as to taper from the at least substantially constant diameter of the flexible, elongate body to a smaller diameter as the distal tip extends distally along a longitudinal axis of the flexible, elongate body.
